(19) United States
(12) Patent Application Publication
(10) Pub. No.: US 2007/0244110 A1
(43) Pub. Date: Oct. 18, 2007

(54) Treatment of prostate cancer, melanoma or hepatic cancer

(75) Inventors: Shinichi Yaguchi, Tokyo (JP); Ichiro Koshimizu, Tokyo (JP); Hisashi Yoshimi, Tokyo (JP); Toshiyuki Matsuno, Tokyo (JP); Tetsuo Watanabe, Tokyo (JP); Yoshio Tsuchida, Tokyo (JP); Kenichi Saitoh, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: ZENYAKU KOGYO KABUSHIKI KAISHA, Chuo-ku (JP)

(21) Appl. No.: 11/404,078

(22) Filed: Apr. 14, 2006

(51) Int. Cl. A61K 31/5377

(52) U.S. Cl. 514/232.8

(57) ABSTRACT

The present application describes a method of treating prostate cancer, melanoma or hepatic cancer in a subject in need thereof, the method comprising administering to said subject a therapeutically effective amount of the heterocyclic compound represented by Formula I or its pharmaceutically acceptable salt:

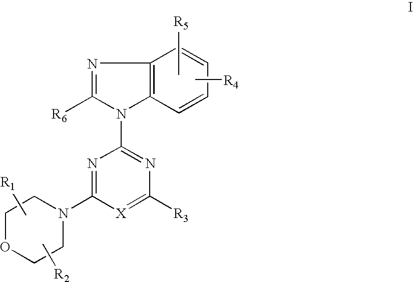

### BACKGROUND ART

1. Field of the Invention

The present invention relates to a method for treating prostate cancer, melanoma or hepatic cancer in a subject.

2. Related Art

s-Triazine (1,3,5-triazine) and pyrimidine derivatives have been researched in the fields of synthetic resins, synthetic fibers, dyes and agricultural chemicals and a number of such compounds have been synthesized. In the field of pharmaceuticals, studies have been performed with respect to antitumor, anti-inflammatory, analgesic and antispasmodic activities. In particular, hexamethylmelamine (HMM) is well-known and has been developed as an analogue of the antitumor agent triethylenemelamine (TEM) [see B. L. Johnson et al. Cancer, 42: 2157-2161 (1978)].

TEM is known as an alkylating agent and is an s-triazine derivative having cytotoxic antitumor activity. HMM has been marketed in Europe as being for the treatment of ovarian and small cell lung cancers, and its action on solid cancers makes it attractive.

Among the s-triazine derivatives, imidazolyl-s-triazine derivatives which exhibit cytotoxic and selective aromatase inhibitory activities have been proposed as medicines for estrogen-dependent diseases such as endometriosis, multicystic ovarium, mastosis, endometrium carcinoma and breast cancer (see PCT publication WO93/17009).

Furthermore, after an extensive study to enhance the antitumor activities of HMM and to reduce the aromatase inhibitory activities of imidazolyl-s-triazine derivatives, the present inventors have found s-triazine and pyrimidine derivatives wherein an imidazole ring is replaced with a benzimidazole ring (see PCT publications WO99/05138 and WO00/43385). After a further study to find compounds having improved the antitumor activities, the inventors found s-triazine and pyrimidine derivatives having a substituted benzimidazole ring (see PCT publications WO02/088112; WO2004/037812 and WO2005/095389).

However, there is still room for improvement of HMM with respect to its antitumor spectrum and intensity of antitumor activities against solid cancers in B. L. Johnson et al. Cancer, 42: 2157-2161 (1978). As to imidazolyl-s-triazine derivatives as disclosed in WO093/17009, they are limited in application since they exhibit considerably higher aromatase inhibitory activities than their cytotoxic activities and application of them to cancerous patients other than those who suffer from estrogen-dependent diseases may lead to development of secondary effects such as menstrual disorders due to lack of estrogen. There is still, therefore, a strong demand for medicines with no aromatase inhibitory activities and effective for solid cancers.

Furthermore, for the compounds described in WO99/05138; WO00/43385; WO02/088112; WO2004/037812; and WO2005/095389, some may require further improvement of their antitumor activities, whereas for others, investigation has been restricted to activities for specific types of cancers such as breast cancer.

After diligent investigation, the present inventors have found that some of the above compounds are also effective for prostate cancer, melanoma or hepatic cancer.

### SUMMARY OF THE INVENTION

In an aspect of the present invention, there is provided a method for treating prostate cancer, melanoma or hepatic cancer in a subject in need thereof, the method comprising administering to the subject a therapeutically effective amount of the heterocyclic compound represented by Formula I: 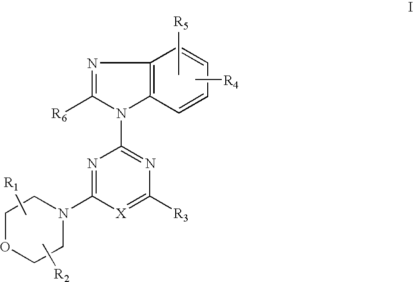
or a pharmaceutically acceptable salt thereof, wherein,
X represents C—R₇ wherein R₇ denotes hydrogen or a halogen;
R₁ and R₂ independently represent hydrogen or a C₁-C₆ alkyl;
R₃ represents:
   an optionally substituted morpholino;
   an optionally substituted pyrrolydinyl; or
   a —NR₁₀R₁₁ group, wherein R₁₀ and R₁₁ independently denote a C₁-C₆ alkyl, a hydroxy C₁-C₆ alkyl, a morpholino with C₁-C₆ alkyl;
R₄ represents hydrogen, a C₁-C₆ alkyl, a C₁-C₆ alkoxy, a C₁-C₆ alkoxycarbonyloxy, amino, or hydroxyl;
R₅ represents hydrogen, a C₁-C₆ alkyl, a halogen, or hydroxyl;
R₆ represents:
   a C₁-C₆ alkyl;
   a hydroxy C₁-C₆ alkyl;
   a —CHₙF₃₋ₙ group wherein n denotes 1 or 2;
   —NHR₁₂, wherein R₁₂ denotes hydrogen or a COR₁₃ group, wherein R₁₃ denotes hydrogen, a C₁-C₆ alkyl or a C₁-C₆ alkoxy.

In an embodiment, R₃ represents morpholino or pyrrolydinyl each optionally substituted with one to four C₁-C₆ alkyl, hydroxy C₁-C₆ alkyl, mono-halogenomethyl or —CH₂NR₈R₉ groups, wherein R₈ denotes hydrogen or a C₁-C₆ alkyl, R₉ denotes hydrogen or a C₁-C₆ alkoxycarbonyl.

In another aspect of the invention, there is provided a method for treating gastric cancer, pancreatic cancer, brain tumor, ovarian cancer, uterine cancer, renal cancer, head and neck cancer, skin cancer, bladder cancer, uroepithelial cancer, osteosarcoma, testicular tumor, rectal cancer, mediastinal tumor, choriocarcinoma, soft tissue sarcoma, thyroid cancer, adrenal cancer, or germ cell tumor in a subject in need thereof, the method comprising administering to the subject a therapeutically effective amount of the heterocyclic compound represented by Formula I as described above.

In a further aspect of the invention, there is provided a method of inhibiting proliferative activity in a prostate cancer, melanoma or hepatic cancer cell as well as gastric cancer, pancreatic cancer, brain tumor, ovarian cancer, uterine cancer, renal cancer, head and neck cancer, skin cancer, bladder cancer, uroepithelial cancer, osteosarcoma, testicular tumor, rectal cancer, mediastinal tumor, choriocarcinoma, soft tissue sarcoma, thyroid cancer, adrenal cancer, or germ cell tumor, the method comprising causing the cancerous cell to contact an effective amount of the heterocyclic compound represented by Formula I as described above.

In yet another aspect, the present invention provides a method for manufacturing an anticancer drug for the treatment of prostate cancer, melanoma or hepatic cancer as well as gastric cancer, pancreatic cancer, brain tumor, ovarian cancer, uterine cancer, renal cancer, head and neck cancer, skin cancer, bladder cancer, uroepithelial cancer, osteosarcoma, testicular tumor, rectal cancer, mediastinal tumor, choriocarcinoma, soft tissue sarcoma, thyroid cancer, adrenal cancer, or germ cell tumor, comprising using one or more compounds represented by Formula I as described above.

These and other features and advantages of the present invention will be described in more detail below.

### DETAILED DESCRIPTION OF THE INVENTION

As used in the present specification, the following terms and words are generally intended to have the meanings as set forth below.

The term “halogen” refers to fluorine, chlorine, bromine or iodine.

The term “C₁-C₆” refers to a group having 1 to 6 carbon atoms unless otherwise indicated.

The “C₁-C₆ alkyl” refers to a straight- or branched-chain alkyl group such as methyl, ethyl, n-propyl, isopropyl, n-butyl, tert-butyl, n-pentyl or n-hexyl.

“Hydroxy C₁-C₆ alkyl” refers to the above-mentioned “C₁-C₆ alkyl” with any of the carbon atoms coupled to a hydroxy group.

“C₁-C₆ alkoxy” refers to a straight- or branched-chain alkoxy group such as methoxy, ethoxy, n-propoxy, isopropoxy, n-butoxy, tert-butoxy, n-pentyloxy or n-hexyloxy.

“Optionally substituted” means that the substitution may or may not occur.

“Pharmaceutically acceptable salt” refers to those that are not biologically acceptable while retaining the pharmacological effectiveness of the free heterocyclic compounds. Preferably, the pharmaceutically acceptable salts are pharmaceutically acceptable acid addition salts which may include, for example, inorganic salts such as hydrochloride, sulfate, hydrobromide, nitrate and phosphate as well as organic acid salts such as acetate, oxalate, propionate, glycolate, lactate, pyruvate, malonate, succinate, maleate, fumarate, malate, tartarate, citrate, benzoate, cinnamate, methanesulfonate, benzenesulfonate, p-toluenesulfonate and salicylate.

“Therapeutically effective amount” refers to an amount of a compound of the invention that is effective to treat a subject suffering from a disorder.

“Subject” as used herein includes humans and other animals such as dogs, cats, rabbits, hamsters, rats, mice, and other organisms. In a preferred embodiment, the subject is a human patient.

“Hepatic cancer” refers to cellular-proliferative pathological states in the liver, and may include hepatoma (hepatocellular carcinoma), cholangiocarcinoma, hepatoblastoma, hepatocellular adenoma, angiosarcoma, hemangioma, or the like. “Melanoma” refers to malignant neoplasm originating from melanocytes. “Prostate cancer” refers to cellular-proliferative pathological states in the prostate, and may include adenocarcinoma or sarcoma.

“Treating” as used herein includes preventing, inhibiting or relieving the pathological state.

The present method of treating prostate cancer, melanoma, hepatic cancer, gastric cancer, pancreatic cancer, brain tumor, ovarian cancer, uterine cancer, renal cancer, head and neck cancer, skin cancer, bladder cancer, uroepithelial cancer, osteosarcoma, testicular tumor, rectal cancer, mediastinal tumor, choriocarcinoma, soft tissue sarcoma, thyroid cancer, adrenal cancer, or germ cell tumor in a subject in need thereof, comprises administering to the subject a therapeutically effective amount of the heterocyclic compound represented by Formula I: 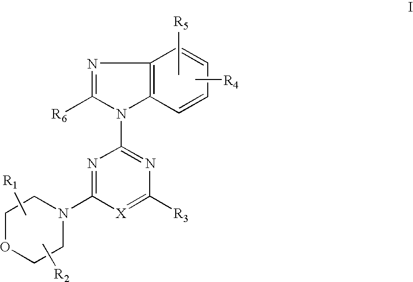
or a pharmaceutically acceptable salt thereof, wherein,
X represents C—R₇ wherein R₇ denotes hydrogen or a halogen;
R₁ and R₂ independently represent hydrogen or a C₁-C₆ alkyl;
R₃ represents:
   an optionally substituted morpholino;
   an optionally substituted pyrrolydinyl; or
   a —NR₁₀R₁₁ group, wherein R₁₀ and R₁₁ independently denote a C₁-C₆ alkyl, a hydroxy C₁-C₆ alkyl, a morpholino with C₁-C₆ alkyl;
R₄ represents hydrogen, a C₁-C₆ alkyl, a C₁-C₆ alkoxy, a C₁-C₆ alkoxycarbonyloxy, amino, or hydroxyl;
R₅ represents hydrogen, a C₁-C₆ alkyl, a halogen, or hydroxyl;
R₆ represents:
   a C₁-C₆ alkyl;
   a hydroxy C₁-C₆ alkyl;
   —CHₙF₃₋ₙ group wherein n denotes 1 or 2;

—NHR₁₂, wherein R₁₂ denotes hydrogen or a COR₁₃ group, wherein R₁₃ denotes hydrogen, a C₁-C₆ alkyl or a C₁-C₆ alkoxy.

In the foregoing, R₃ represents morpholino or pyrrolydinyl each optionally substituted with one to four C₁-C₆ alkyl, hydroxy C₁-C₆ alkyl, mono-halogenomethyl or —CH₂NR₈R₉ groups, wherein R₈ denotes hydrogen or a C₁-C₆ alkyl, R₉ denotes hydrogen or a C₁-C₆ alkoxycarbonyl.

The compounds of the present invention may have asymmetric carbon atoms in the structure. It is to be understood that isomers due to such asymmetric carbon atoms or combination (racemate) of any of the isomers are included in the category of the compounds according to the present invention.

As the compounds of the invention, mention can be preferably made of the following compounds:
2-(2-difluoromethylbenzimidazol-1-yl)-4,6-dimorpholino-1,3,5-triazine (Compound 1);
2-(2-difluoromethylbenzimidazol-1-yl)-4-(2,2-dimethylmorpholino)-6-morpholino-1,3,5-triazine (Compound 2);
2-(2-difluoromethylbenzimidazol-1-yl)-4-(trans-2,5-dimethylmorpholino)-6-morpholino-1,3,5-triazine (Compound 3);
2-(2-difluoromethyl-4-methoxybenzimidazol-1-yl)-4,6-dimorpholino-1,3,5-triazine (Compound 4);
2-(2-difluoromethyl-4-methoxybenzimidazol-1-yl)-4-(2,2-dimethylmorpholino)-6-morpholinopyrimidine (Compound 5);
2-(2-difluoromethyl-4-methoxybenzimidazol-1-yl)-4-(cis-2,6-dimethylmorpholino)-6-moropholinopyrimidine (Compound 6);
2-(2-difluoromethyl-4-hydroxybenzimidazol-1-yl)-4-(2-hydroxymethylpyrrolidin-1-yl)-6-morpholinopyrimidine (Compound 7);
2-(2-difluoromethyl-4-hydroxybenzimidazol-1-yl)-4,6-dimorpholino-1,3,5-triazine (Compound 8);
2-(2-difluoromethyl-4-hydroxybenzimidazol-1-yl)-4-(2-hydroxymethylpyrrolidin-1-yl)-6-morpholino-1,3,5-triazine (Compound 9);
2-(2-difluoromethyl-4-hydroxybenzimidazoly-1-yl)-4-(3,3-dimethylmoropholino)-6-morpholinopyrimidine (Compound 10);
2-(2-difluoromethyl-4-hydroxybenzimidazol-1-yl)-4-(cis-2,6-dimethylmorpholino)-6-morpholino-1,3,5-triazine (Compound 11);
2-(6-amino-4-chloro-2-difluoromethylbenzimidazol-1-yl)-4-(2,2-dimethyl-morpholino)-6-morpholino-1,3,5-triazine (Compound 12);
2-(2-difluoromethyl-4-acetoxybenzimidazoly-1-yl)-4-(cis-2,6-dimethyl-moropholino)-6-morpholinopyrimidine (Compound 13);
2-(2-difluoromethyl-4-methoxycarbonyloxybenzimidazoly-1-yl)-4-(3,3-dimethylmoropholino)-6-morpholinopyrimidine (Compound 14);
2-(4-ethoxycarbonyloxy-2-difluoromethylbenzimidazoly-1-yl)-4-(cis-2,6-dimethylmoropholino)-6-morpholinopyrimidine (Compound15).

The compounds of the invention are already known from U.S. Pat. No. 6,251,900 corresponding to WO99/05138; U.S. Patent Application 2004/116,421 A1 corresponding to WO02/088112; U.S. Patent Application 2006/009,440A1 corresponding to WO2004/037812; and WO2005/095389, the disclosure of each of these applications being explicitly incorporated herein by reference.

The compounds of the invention or their pharmaceutically acceptable salts can be administered in pure form but usually in an appropriate pharmaceutical composition via any of the accepted routes of administration. Thus, the administration can be orally or parenterally, e.g., intravenously, intramuscularly, or subcutaneously. For oral administration, the compound or composition may be in solid or liquid dosage forms, such as tablets, coated tablets, powders, granules, capsules, microcapsules, syrups and the like. For parenteral administration, the compound or composition may be in the forms of solutions, suspensions, inhalants, ophthalmic formulations, suppository, poultices and the like.

The pharmaceutical compositions may include, in addition to the compound of the invention, a conventional pharmaceutically acceptable carrier, excipient or diluent, and further may include other ingredients such as binders, lubricants, disintegrators, emulsifiers, preservatives, stabilizers and dispersing agents such as lactose, sucrose, starch, dextrin, crystalline cellulose, kaolin, calcium carbonate, talc, or magnesium stearate.

The compounds of the invention or their pharmaceutical acceptable salts are administered in a therapeutically effective amount that may vary depending upon various factors including the type of the cancer to be treated, the severity of the cancer, the age, body weight, general health, route of administration, drug combination, and the undergoing therapy and the like. A daily dosage for an adult may be in the range of from 100 to 1,000 mg and may be given in divided doses two or three times a day.

### EXAMPLES

The present invention will be more fully described by way of the following examples which in no way serve to limit the scope of the invention, but rather are presented for illustrative purposes.

### Examples of Synthesis of Compounds

Some of the heterocyclic compounds for use in the methods of the present invention were prepared according to the procedures described in prior applications or alternatively according to synthetic techniques well known in the art.

### Example 1

### Synthesis of 2-(2-difluoromethylbenzimidazol-1-yl)-4,6-dimorpholino-1,3,5-triazine (Compound 1):

(1) After a mixture of o-phenylenediamine (51.9 g, 480 mmol), difluoroacetic acid (50.7 g, 528 mmol) and 4N HCl (500 mL) was refluxed for 2 hours, the reaction mixture was neutralized by the careful addition of anhydrous sodium carbonate. The resulting precipitates were collected and washed with water to give 2-difluoromethyl-1H-benzimidazole (72.2 g) as white crystal in 90% yield.

MS m/z: 168(M⁺)

¹H-MNR (CDCl₃) δ: 6.91 (1H, t, J=54 Hz), 7.36-7.53(2H, m), 7.56 (1H, d, J=7 Hz), 7.86 (1H, d, J=7 Hz).

(2) To a solution of cyanuric acid (230.5 g, 1,250 mmol) in acetone (2.25 L) was added dropwise a solution of morpholine (98.0 g, 1,125 mmol) and triethylamine (113.0g, 1,125 mmol) in acetone (2.25 L) at 5° C. The resulting mixture was poured into water. White precipitates were collected and washed with methanol to give 2,4-dichloro-6-morpholino-1,3,5-triazine (231.9 g, 986.9 mmol) as white crystal in 88% yield.

MS m/z: 234(M⁺)

¹H-MNR(CDCl₃) δ: 3.75 (4H, t, J=4 Hz), 3.87 (4H, t, J=4 Hz).

(3) A mixture of 2-difluoromethyl-1H-benzimidazole (72.2 g) (67.0 g, 400 mmol), 2,4-dichloro-6-morpholino-1,3,5-triazine (94.0 g, 400 mmol) and anhydrous potassium carbonate (221.1 g, 1600 mmol) in DMF (1.60 L) was stirred at room temperature for 4 hours. The resulting mixture was poured into water. White precipitates were collected and washed with DMF and then acetone successively. The solid was dried under reduced pressure to give 4-chrolo-2-(2-difluoromethylbenzimidazol-1-yl)-6-morpholino-1,3,5-triazine (131.5 g, 358.6 mmol) as white crystal in 90% yield.

MS m/z: 366 (M⁺)

¹H-MNR(CDCl₃) δ: 3.80-3.87 (4H, m), 3.94-4.01 (4H, m), 7.38-7.53 (2H, m), 7.58 (1H, t, J=54 Hz), 7.90 (1H, d, J=7 Hz), 8.42 (1H, d, J=7 Hz).

(4) A solution of 4-chrolo-2-(2-difluoromethylbenzimidazol-1-yl)-6-morpholino-1,3,5-triazine (102.7 g, 280 mmol) in morpholine (871 g) was stirred at 70° C. for 2 hours. The resulting mixture was poured into water. White precipitates were collected and recrystallized by ethanol/acetone to give 2-(2-difluoromethylbenzimidazol-1-yl)-4,6-dimorpholino-1,3,5-triazine (112.5 g, 270 mmol) in 96% yield.

Mp: 211-214° C.

MS m/z: 417 (M⁺)

¹H-MNR(CDCl₃) δ: 3.79-3.81 (8H, m), 3.87-4.00 (8H, m), 7.37-7.45 (2H, m), 7.57 (1H, t, J=54 Hz), 7.90 (1H, d, J=7 Hz), 8.33 (1H, d, J=7 Hz).

### Example 2

The following Compounds 2-3 were prepared analogously to the examples of U.S. Patent Application 2004/116,421A1 corresponding to WO02/088112 from the appropriate starting materials, whereas Compounds 4-6 were prepared analogously to the examples of WO2005/095389 from the appropriate starting materials.

2-(2-difluoromethylbenzimidazol-1-yl)-4-(2,2-dimethylmorpholino)-6-morpholino-1,3,5-triazine (Compound 2):

Mp: 176-178 ° C.

NMR(CDCl₃) δ: 1.29 (6H, s), 3.6-3.9 (14H, m), 7.3-8.0 (4H, m), 8.3-8.4 (1H, m) MS m/z: 445(M⁺)

2-(2-difluoromethylbenzimidazol-1-yl)-4-(trans-2,5-dimethylmorpholino)-6-morpholino-1,3,5-triazine (Compound 3):

Mp: 166-169° C.

NMR(CDCl₃) δ: 1.31 (3H, d, J=7 Hz), 1.39 (3H, d, J=7 Hz), 3.4-4.3 (13H, m), 4.6-4.8 (1H, m), 7.3-7.5(2H, m), 7.58 (1H, t, J=7 Hz), 7.8-8.0 (1H, m), 8.2-8.3 (1H, m) MS m/z: 445(M⁺)

2-(2-difluoromethyl-4-methoxybenzimidazol-1-yl)-4,6-dimorpholino-1,3,5-triazine (Compound 4):

Mp: >250° C.

NMR(CDCl₃) δ: 3.5-3.9(16H, m), 4.0(3H, s), 6.82(1H, d, J=7 Hz), 7.35(1H, t, J=7 Hz), 7.48(1H, t, J=53 Hz), 7.88(1H, d, 7 Hz)

MS m/z : 447(M⁺)

2-(2-difluoromethyl-4-methoxybenzimidazol-1-yl)-4-(2,2-dimethylmorpholino)-6-morpholinopyrimidine (Compound 5)

Mp : 160-162 ° C.

NMR (CDCl₃) δ: 1.30 (6H, s), 3.49 (2 H, s), 3.6-3.9 (12 H, m), 4.04 (3H, s), 5.47 (1H, s), 6.79 (1H, d, J=8 Hz), 7.32 (1H, t, J=8 Hz), 7.41 (1H, t, J=53 Hz,), 7.78 (1H, d, J=8 Hz,).

MS m/z 474 (M⁺).

2-(2-difluoromethyl-4-methoxybenzimidazol-1-yl)-4-(cis-2,6-dimethylmorpholino)-6-moropholinopyrimidine (Compound 6):

Mp: 180-181 ° C.

NMR(CDCl₃) δ:1.28 (6H, d, J=6 Hz), 2.6-2.7(2H, m), 3.6-3.7 (6H, m), 3.80-3.86 (4H, m), 4.04 (3H, s), 4.10-4.14 (2H, m), 5.49 (1H, s), 6.78 (1H, d, J=8 ), 7.32 (1H, d, J=8 Hz), 7.41 (1H, t, J=52 Hz), 7.77 (1H, d, J=8 Hz)

MS m/z : 474(M⁺)

### Example 3

### Synthesis of 2-(2-difluoromethyl-4-hydroxybenzimidazol-1-yl)-4-(2-hydroxy-methylpyrrolidin-1-yl)-6-morpholinopyrimidine (Compound 7)

(1) 2,4,6-Trichloropyrimidine (0.91 g, 5.0 mmol) and potassium carbonate (0.55 g) were added successively to the solution of 4-tert-butyldimethylsilyloxy-2-difluoromethylbenzimidazol (1.49 g, 5.0 mmol) in DMF(10 ml) at room temperature and stirred for 5 hrs. Water was added to the reaction mixture and extracted with ethyl acetate a few times. The combined extracts were washed with brine and dried over anhydrous magnesium sulfate. The solution was evaporated under reduced pressure. The residue was columnchromatographed on a silica gel using n-hexane-ethyl acetate (8:1) to give 2-(4-tert-butyldimethylsilyloxy-2-difluoromethylbenzimidazol-1-yl)-4,6-dichloropyrimidine (1.12g) in yield 50%.

(2) 2-Pyrrolidinemethanol (0.13 ml, 1.3 mmol) and potassium carbonate (179 mg) were added successively to the solution of 2-(4-tert-butyldimethylsilyloxy-2-difluoromethylbenzimidazol-1-yl)-4,6-dichloropyrimidine (386 mg, 0.87 mmol) in DMF (6 ml) at room temperature and stirred for 0.5 hr. Water was added to the reaction mixture and extracted with ethyl acetate a few times. The combined extracts were washed with brine and dried over anhydrous magnesium sulfate and then evaporated under reduced pressure. The residue was chromatographed on a silica gel using n-hexane-ethyl acetate(1:1) to give 2-(4-tert-butyldimethylsilyloxy-2-difluoromethylbenzimidazol-1-yl)-4-(2-hydroxymethylpyrrolidin-1-yl)-6-chloropyrimidine(291mg) in yield 64%.

(3) 2-(4-Tert-butyldimethylsilyloxy-2-difluoromethylbenzimidazol-1-yl)-4-(2-hydroxymethylpyrrolidin-1-yl)-6-chloropyrimidine (281 mg, 0.54 mmol) was added to morpholine (4.4 g, 50 mmol) and stirred at room temperature for 9 hrs. Water was added to the reaction mixture and extracted with ethyl acetate a few times. The combined extracts were washed with brine and dried over anhydrous magnesium sulfate and then evaporated in vaccuo. The residue was chromatographed on a silica gel using n-hexane-ethyl acetate (2:3) to give 2-(4-tert-butyldimethylsilyloxy-2-difluoromethyl-benzimidazol-1-yl)-4-(2-hydroxymethylpyrrolidin-1-yl)-6-morpholinopyrimidine (216mg) in yield 72%.

(4) Tetra-n-butylammoniumfluoride (0.4 ml:1 M in THF solution) was added to the solution of 2-(4-tert-butyldimethylsilyloxy-2-difluoromethylbenzimidazol-1-yl)-4-(2-hydroxymethylpyrrolidin-1-yl)-6-morpholinopyrimidine (213 mg, 0.38 mmol) in anhydrous THF (7 ml) at room temperature and stirred for 0.5 hr. Water was added to the reaction mixture and extracted with ethyl acetate a few times. The combined extracts was washed with brine and dried over anhydrous magnesium sulfate and then evaporated under reduced pressure. The residue was chromatographed on a silica gel using n-hexane-ethyl acetate (1:4) to give 2-(2-difluoromethyl-4-hydroxybenzimidazol-1-yl)-4-(2-hydroxymethylpyrrolidin-1-yl)-6-morpholinopyrimidine (101 mg) in yield 60%.

Mp: 95-198° C.

NMR (CDCl₃) δ: 2.0-2.1 (4H, m), 3.4-4.0 (12H, m), 4.0-4.1 (1H, m), 4.3-4.4 (1H, m), 5.36 (1H, s), 6.85 (1H, d, J=8 Hz), 7.28 (1H, t, J=8 Hz), 7.58 (1H, brs), 7.58 (1H, t, J=54 Hz), 7.73 (1H, d, J=8 Hz).

MS m/z: 446(M⁺)

### Example 4

The following Compounds 8 and 10-11 were prepared analogously to the examples of U.S. Patent Application 2004/116,421A1 corresponding to WO02/088112, and Compounds 9 and 12 were prepared analogously to the examples of U.S. Patent Application 2006/009,440A1 corresponding to WO2004/037812, from the appropriate starting materials.

2-(2-difluoromethyl-4-hydroxybenzimidazol-1-yl)-4,6-dimorpholino-1,3,5-triazine (Compound 8):

Mp: >250° C.

NMR(DMSO-d₆) δ: 3.70-3.90 (16H, m), 6.76 (1H, d, J=8 Hz), 7.73 (1H, t, J=8 Hz), 7.70(1H, t, J=54 Hz), 7.74 (1H, d, 8 Hz), 10.24 (1H, brs)

MS m/z : 433(M⁺)

2-(2-difluoromethyl-4-hydroxybenzimidazol-1-yl)-4-(2-hydroxymethylpyrrolidin-1-yl)-6-morpholino-1,3,5-triazine (Compound 9):

Mp: 245° C. (dec.)

NMR(CDCl₃) δ: 1.9-2.1 (4H, m), 3.5-4.0 (12H, m), 4.7-4.8 (1H, m), 5.1-5.3 (1H, m), 6.89 (1H, d, J=9 Hz), 7.30 (1H, t, J=9 Hz), 7.50 (1H, brs), 7.55 (1H, t, J=54 Hz), 7.83 (1H, d, J=9 Hz).

MS m/z: 447(M⁺)

2-(2-difluoromethyl-4-hydroxybenzimidazol-1-yl)-4-(3,3-dimethylmoropholino)-6-morpholinopyrimidine (Compound 10):

Mp: 204-206° C.

NMR(CDCl₃) δ: 1.48 (6H, s), 3.50 (2H, s), 3.6-3.8 (6H, m), 3.8-4.0 (6H, m), 5.76 (1H, s), 6.68(1H, d, J=7 Hz), 7.29 (1H, d, J=7 Hz), 7.49 (1H, t, J=54 Hz), 7.66 (1H, d, 7 Hz)

MS m/z : 460(M⁺)

2-(2-difluoromethyl-4-hydroxybenzimidazol-1-yl)-4-(cis-2,6-dimethylmorpholino)-6-morpholino-1,3,5-triazine (Compound 11):

Mp: 214-215° C.

NMR(CDCl₃-d₁) δ: 1.29 (6H, d, J=6 Hz), 2.60-2.80 (2H, m), 3.60-3.80 (2H, m), 3.80-4.00 (8H, m), 6.71 (1H, brs), 6.90 (1H, d, J=7 Hz), 7.32 (1H, t, J=7 Hz), 7.55 (1H, t, J=54 Hz), 7.80 (1H, d, 7 Hz)

MS m/z : 461(M⁺)

### Example 5

### Synthesis of 2-(6-amino-4-chloro-2-difluoromethylbenzimidazol-1-yl)-4-(2,2-dimethylmorpholino)-6-morpholino-1,3,5-triazine (Compound 12):

(1) 2,4-Dichloro-6-morpholino-1,3,5-triazine (542 mg, 2.3 mmol) and potassium carbonate (500 mg) were added successively to the solution of 6-amino-4-chloro-2-difluoromethylbenzimidazole (500 mg, 2.3mmol) in acetone (50 ml) at −15° C. under stirring and continued stirring at room temperature for 5 hrs. The solvent was removed under reduced pressure and the residue was chromatographed on a silica gel using n-hexane-ethyl acetate (1:4) to give 2-(6-amino-4-chloro-2-difluoromethylbenzimidazol-1-yl)-4-chloro-6-morpholino-1,3,5-triazine (272mg) in yield 28%.

(2) 2,2-Dimethylmorpholine hydrochloride (150 mg, 1.0 mmol) and potassium carbonate (500 mg) were added successively to the solution of 2-(6-amino-4-chloro-2-difluoromethylbenzimidazol-1-yl)-4-chloro-6-morpholino-1,3,5-triazine (150 mg, 0.36 mmol) in DMF (6 ml) at −15° C. under stirring and continued stirring over night. Water was added to the reaction mixture and extracted with ethyl acetate a few times. The combined extracts were washed with brine and dried over anhydrous magnesium sulfate and evaporated under reduced pressure. The residue was chromatographed on a silica gel using n-hexane-ethyl acetate (1:2) to give 2-(6-amino-4-chloro-2-difluoromethylbenzimidazol-1-yl)-4-(2,2-dimethylmorpholino)-6-morpholino-1,3,5-triazine (130 mg) in yield 73%.

Mp: 238 ° C. (dec.)

NMR (CDCl₃) δ: 1.27(6H, s), 3.68 (2H, s), 3.7-3.9 (12H, m), 6.82 (1H, d, J=2.3 Hz), 7.42 (1H, dt, J=9.6 Hz, J=53 Hz), 7.50 (1H, d, J=2.3 Hz)

MS m/z:494(M⁺)

### Example 6

The following compounds were prepared analogously to the examples disclosed in prior applications or alternatively according to synthetic techniques well known in the art.

2-(2-difluoromethyl-4-acetoxybenzimidazol-1-yl)-4-(cis-2,6-dimethylmoropholino)-6-morpholinopyrimidine (Compound 13):

Mp: 181-183° C.

NMR (CDCl₃) δ: 1.28 (6H, d, J=6 Hz), 2.47 (3H, s), 2.6-2.8(2H, m), 3.6-4.2(12H, m), 5.49(1H, s), 7.14(1H, d, J=8 Hz), 7.39(1H, t, J=8 Hz), 7.43 (1H, t, J=53 Hz), 8.07(1H, d, MS m/z 502 (M⁺)

2-(2-difluoromethyl-4-methoxycarbonyloxybenzimidazol-1-yl)-4-(2,2-dimethyl-moropholino)-6-morpholinopyrimidine (Compound 14):

Mp: 172-175° C.

NMR (CDCl₃) δ: 1.30 (6H, s), 3.49 (2 H, s), 3.5-4.0 (12H, m), 3.96 (3H, s), 5.48 (1H, s), 7.22 (1H, d, J=8 Hz), 7.39 (1H, t, J=8 Hz), 7.43 (1H, t, J=53 Hz,), 8.09 (1H, d, J=8 Hz,).

MS m/z 518 (M⁺).

2-(4-ethoxycarbonyloxy-2-difluoromethylbenzimidazol-1-yl)-4-(cis-2,6-dimethyl-moropholino)-6-morpholinopyrimidine (Compound 15):

Mp: 178-180° C.

NMR (CDCl₃) δ: 1.28 (6H, s), 1.42 (3H, t, J=7 Hz), 2.6-2.8 (2H, m), 3.5-4.2 (12H, m), 4.37 (2H, q, J=7 Hz), 5.50(1H, s), 7.22 (1H, d, J=8 Hz), 7.39 (1H, t, J=8 Hz), 7.43 (1H, t, J=53 Hz,), 8.08(1 H, d, J=8 Hz,).

MS m/z 532 (M⁺).

### ASSAY EXAMPLES

Hereafter, the pharmacological effects of the heterocyclic compounds I of the invention will be described. In the text below, the compound numbers indicated in the tested compounds correspond to the numbers of the compounds listed above.

### Assay Example 1

### In Vitro Anti-Tumor Experiment

Hepatoma HepG2 cells were cultured in a MEM culture medium (containing 10% fetal bovine serum, 25 mM HEPES and 0.1 mg/ml kanamycin) under conditions of 5% carbon dioxide at 37° C. HepG2 cells were harvested as single free cells by a tripsin/EDTA treatment in the logarithmic growth phase, and a cell suspension solution consisted of 2×10⁴ cells per ml was prepared with MEM culture medium (containing 10% fetal bovine serum, 25 mM HEPES and 0.1 mg/ml kanamycin). The test compound was dissolved in DMSO, then diluted with RPMI1640 culture medium (containing 10% fetal bovine serum, 25 mM HEPES, 0.1 mg/ml kanamycin), and adjusted to a concentration of 2.0×10⁻⁹ to 2.0×10⁻³ M.

0.1 ml of the cell suspension solution was put in each well of a 96-well plate, then cultured for 24 hours to make the cells adhere to the microplates, and 0.1 ml of the test compound was added in each well and incubated for 72 hours under conditions of 5% carbon dioxide at 37° C. The 50% growth inhibition concentration (GI₅₀ value μM) was computed from the growth inhibition rates at various test sample concentrations, and the results are shown in Table 1.

When using cells other than HepG2 cells, the following culture media were used instead of the MEM culture medium, and the following single cell suspensions were prepared with the following media:

PC-3 prostate cancer cells:

F12K culture medium containing 10% fetal bovine serum, 2×10⁴ cell suspension

B16F10 melanoma cells:

RPMI1640 culture medium containing 10% fetal bovine serum, 1×10⁴ cell suspension

TABLE 1

| | GI₅₀ Values of Cells (μM) | | |
|---|---|---|---|
| | Hepatoma | Prostate Cancer | Melanoma |
| Compound No. | (HepG2) | (PC-3) | (B16F10) |
| 1 | 1.15 | 0.24 | 0.31 |
| 2 | 1.5 | 0.74 | 0.95 |
| 3 | 1.12 | 0.49 | 1.14 |
| 4 | 0.34 | 0.19 | 0.25 |
| 6 | 0.89 | 0.54 | 1.21 |
| 8 | 0.12 | 0.01 | 0.018 |
| 9 | 0.15 | 0.02 | 0.063 |
| 10 | 0.09 | 0.02 | 0.042 |
| 12 | 0.26 | 0.10 | 0.27 |

### Assay Example 2

### In Vivo Anti-Tumor Experiment

Human tumor xenografts were generated with PC-3 prostate cancer cells, and WiDr colon cancer cells, and were grown as subcutaneous tumors in mutant BALB/c nude mice. Each nude mouse was subcutaneously inoculated with a tumor fragment of 2 mm by 2 mm by 2 mm. When tumors reached a volume of 100-300 mm³, animals were divided randomly into test groups of five mice (day 0). The samples were orally administered at a dosage of 100, 200 or 400 mg/kg once daily, 6 days a week for two weeks. The length (L) and width (W) of the subcutaneous tumor mass were measured by calipers in live mice, and the tumor volume (TV) was calculated as: TV=(L×W²)/2. The tumor volume at day n was expressed as relative tumor volume (RTV), according to the formula RTV=TVₙ/TV₀, where TVₙ is the tumor volume at day n, and TV₀ is the tumor volume at day 0. T/C% on day 14 was determined by calculating RTV as: T/C%=100×(mean RTV of treated group)/(mean RTV of control group). The specimens in which T/C (%) was 50% or less and having a significant difference at P value 1% on one side in a Mann-Whitney U test were evaluated as effective (+). As a result, the compounds of the present invention were effective in vivo, for the treatment of prostate cancer, and even more effective than the treatment of colon cancer.

TABLE 2

| | T/C (%) | | |
|---|---|---|---|
| | | | Colon Cancer |
| Compound No. | Dosage (mg/kg) | Prostate Cancer | (reference) |
| 1 | 400 | +, 24.8 | +, 33.2 |
| 5 | 100 | Not done | + |
| | 200 | +, 39.1 | Not done |
| 6 | 100 | +, 32.3 | +, 24.8 |
| | 200 | +, 16.2 | +, 29.5 |
| 9 | 200 | +, 31.8 | +, 49.6 |
| 10 | 200 | +, 38.1 | +, 46.2 |
| | 400 | +, 20.8 | −, 63.1 |
| 11 | 200 | −, 66.5 | −, 85.4 |
| | 400 | +, 44.3 | −, 83.5 |
| 13 | 200 | −, 65.4 | Not done |
| 14 | 200 | +, 47.3 | Not done |
| 15 | 200 | +, 47.5 | Not done |

What is claimed is:

1. A method of treating prostate cancer, melanoma or hepatic cancer in a subject in need thereof, said method comprising administering to said subject a therapeutically effective amount of the heterocyclic compound represented by Formula I: 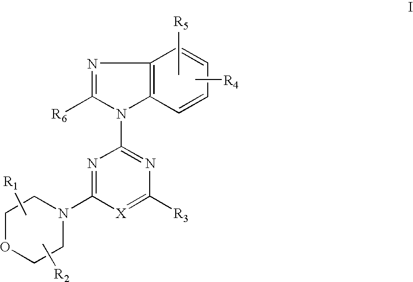
or a pharmaceutically acceptable salt thereof, wherein,
X represents C—R₇ wherein R₇ denotes hydrogen or a halogen;
R₁ and R₂ independently represent hydrogen or a C₁-C₆ alkyl;
R₃ represents:
an optionally substituted morpholino;
an optionally substituted pyrrolydinyl; or
a —NR₁₀R₁₁ group, wherein R₁₀ and R₁₁, independently denote a C₁-C₆ alkyl, a hydroxy C₁-C₆ alkyl, a morpholino with C₁-C₆ alkyl;
R₄ represents hydrogen, a C₁-C₆ alkyl, a C₁-C₆ alkoxy, a C₁-C₆ alkoxycarbonyloxy, amino, or hydroxyl;
R₅ represents hydrogen, a C₁-C₆ alkyl, a halogen, or hydroxyl;
R₆ represents:
a C₁-C₆ alkyl;
a hydroxy C₁-C₆ alkyl;
a —CHₙF₃₋ₙ group wherein n denotes 1 or 2;
—NHR₁₂, wherein R₁₂ denotes hydrogen or a COR₁₃ group, wherein R₁₃ denotes hydrogen, a C₁-C₆ alkyl or a C₁-C₆ alkoxy.

2. The method according to claim 1, wherein R₃ represents morpholino or pyrrolydinyl each optionally substituted with one to four C₁-C₆ alkyl, hydroxy C₁-C₆ alkyl, mono-halogenomethyl or —CH₂NR₈R₉ groups, wherein R8 denotes hydrogen or a C₁-C₆ alkyl, R₉ denotes hydrogen or a C₁-C₆ alkoxycarbonyl.
